(19) United States
(12) Patent Application Publication
(10) Pub. No.: US 2006/0188449 A1
(43) Pub. Date: Aug. 24, 2006

(54) Topical aerosol foams

(76) Inventors: Jane Hirsh, Wellesley, MA (US); John C. Willis II, Mansfield, MA (US); Mark Hirsh, Wellesley, MA (US)

Correspondence Address: PATREA L. PABST;PABST PATENT GROUP LLP, 400 COLONY SQUARE, SUITE 1200, ATLANTA, GA, 30361, US

(21) Appl. No.: 10/565,346

(22) PCT Filed: Oct. 4, 2004
(86) PCT No.: PCT/US04/32714

§ 371 Date: Jan. 20, 2006

Related U.S. Application Data
(60) Provisional application No. 60/508,495, filed on Oct. 3, 2003.

(51) Int. Cl. A61K 31/573; A61L 9/04; A61K 31/56; A61K 31/24

(52) U.S. Cl. 424/45; 514/171; 514/537

(57) ABSTRACT

A stable topical aerosol foam is provided. The foam-forming formulation includes a HFA propellant and an active agent in an emulsion. The emulsion has an oil phase and an aqueous, i.e. water-containing, phase. The active agent may be present in either phase or dispersed in the emulsion. The oil phase may consist at least in part of the HFA propellant. Either or both of the oil phase and the aqueous phase may contain one or more surfactants, emulsifiers, emulsion stabilizers, buffers, and other excipients. In an alternative embodiment, the aqueous phase contains a water-soluble active agent, for example, a local anesthetic, and the oil phase contains a water-insoluble second active agent. The foam is stable on the skin, for example for at least 10 minutes at body temperature, and will disappear into the skin upon rubbing or after prolonged standing. The formulation has the advantage of an inert non-flammable hydrofluorocarbon propellant without requiring the use of additional co-solvents or co-propellants. The composition is administered to the skin or mucous membranes.

### CROSS-REFERENCE TO RELATED APPLICATIONS

This application claims benefit under 35 U.S.C. 119, to U.S. Provisional Application Nos. 60/508,495 entitled “Topical Aerosol Foams”, filed on Oct. 3, 2003, by Mark Hirsh and 60/560,890 entitled “Non-Flammable Topical Aerosol Spray” filed on Apr. 9, 2004, by Jane Hirsh and Donald L. Tibbetts.

### BACKGROUND OF THE INVENTION

Pharmaceutical foams are pressurized dosage forms containing one or more active ingredients that, upon valve actuation, emit a fine dispersion of liquid and/or solid materials in a gaseous medium. Foam formulations are generally easier to apply, are less dense, and spread more easily than other topical dosage forms. Foams may be formulated in various ways to provide emollient or drying functions to the skin, depending on the formulation constituents. Therefore, this delivery technology should be a useful addition to the spectrum of formulations available for topical use; however, as yet, only a few are commercially available. The most convincing argument for the use of foams is ease of use by the patient, and consumer acceptance. Most foam dosage forms used in dermatology have incorporated corticosteroids to date, although some products have also been used to deliver antiseptics, antifungal agents, anti-inflammatory agents, local anesthetic agents, skin emollients, and protectants (American Journal of Drug Delivery, 2003, vol. 1(1), pp. 71-75).

There is growing interest in converting treatments to aerosol foam or mousse formulations, which better penetrate the skin, provide faster treatment and do not leave any greasy residue on skin or clothing compared with conventional ointments. Until now, the most common gas propellant used in aerosol products is chlorofluorocarbon (CFC), an ozone-depleting agent. The Montreal Protocol international treaty signed by 180 nations, banes the use of chlorofluorocarbons (CFCs) as aerosol propellants and mandates the phasing out of CFC agents. No new or revised aerosol formulations may contain CFC propellants, alternative propellants must be used that are more environmentally friendly. Therefore, manufacturers must reformulate or modify existing products to use non-CFC propellants, while maintaining important aspects of the previous formulation, such as accuracy of delivery, stability, etc. The primary CFC substitute is the gas propellant known as hydrofluoroalkane, or HFA.

Although hydrocarbon propellants (due to their minimal ozone depletion effect) can be used in manufacturing of pharmaceutical foams, these propellants are not suited for human use since they are flammable. Just as is the case with CFC propellants, hydrofluoroalkanes (HFAs) that possess high chemical stability can be used as a primary substitute for hydrocarbons. Examples of HFAs are 1,1,1,2,3,3,3-heptafluoropropane (HFA-134a) and 1,1,1,2-tetrafluoroethane (HFA-227). Hydrofluoroalkanes (HFAs) are also often referred to as hydrofluorocarbons (HFCs) and these terms are used interchangeably.

Since replacing a component of any formulation means introducing new properties, and HFAs differ in their solvating power from CFCs and hydrocarbons, providing reproducible performance of reformulated aerosols for pharmaceutical uses represents a challenging task. Often a co-solvent (such as ethanol) needs to be incorporated into the formulation in order to produce a stable product (Pharmaceutical Aerosols, June 2003, p. 21). Such formulations, however, have a number of undesirable aspects. Alcohol co-solvents can dry and irritate the skin. U.S. Pat. No. 6,126,920 suggests that the use of alcohol co-solvents can lead to the burning, itching, and irritation observed in the use of topical foam for delivering betamethasone. Further, volatile alcohols are highly irritating to mucous membranes.

Formulations that contain volatile alcohols as well as alkanes are potential safety hazards due to the high flammability of the product. Moreover, the flammability characteristics of the product require expensive precautions during manufacturing, and may require controlled environments for storage and for disposal of containers after use. For example, WO 85/01876 describes the fire hazards associated with alcohol and alkane containing aerosol foam formulations.

It is therefore an object of the invention to provide alcohol-free topical foam aerosol formulations that use hydrofluoroalkanes (HFAs) as the propellant.

### BRIEF SUMMARY OF THE INVENTION

A stable topical alcohol-free aerosol foam is provided. The foam-forming formulation includes a BFA propellant and an active agent in an emulsion. The emulsion has an oil phase and an aqueous, i.e. water-containing, phase. The active agent may be present in either phase or dispersed in the emulsion. The oil phase may consist at least in part of the HFA propellant. Either or both of the oil phase and the aqueous phase may contain one or more surfactants, emulsifiers, emulsion stabilizers, buffers, and other excipients. In an alternative embodiment, the aqueous phase contains a water-soluble active agent, for example, a local anesthetic, and the oil phase contains a water-insoluble second active agent. The foam is stable on the skin, for example, for at least 10 minutes at body temperature, and disappears into the skin upon rubbing or after prolonged standing.

The formulation has the advantage of including an inert non-flammable hydrofluorocarbon propellant without requiring the use of additional co-solvents or co-propellants. The composition is administered as a metered dose that can be applied to the skin or mucous membranes.

### DETAILED DESCRIPTION OF THE INVENTION

It has been discovered, based on studies with a hydrocortisone acetate topical aerosol foam, that a hydrocarbon propellant can be replaced with an HFA propellant without any other changes to the formulation. Importantly, no ethanol was added to the formulation. This was due to the fact that a predominantly aqueous, 86% (w/w) water, drug-containing emulsion was used to prepare the hydrocortisone acetate topical aerosol foam. Two other structurally and functionally different drugs (lidocaine and itraconazole) were similarly formulated and it was found that, in fact, stable alcohol-free HFA aerosol foam formulations can be obtained when predominantly aqueous drug-containing emulsions were used.

I. Formulation

a. Propellants

The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

b. Active Agents

The active agent may be any material that has a desired effect when applied topically to a mammal, particularly a human. Suitable classes of active agents include anti-inflammatory agents, topical anesthetics, topical antibiotics including anti-fungal agents, and combinations thereof.

The anti-inflammatory agent can be a corticosteroid or a non-steroidal anti-inflammatory drug (NSAID). Suitable corticosteroids include alclometasone dipropionate, amcinonide, beclametasone dipropionate, betamethasone benzoate, betamethasone dipropionate, betamethasone valerate, budesonide, clobetasol propionate, clobetasone butyrate, desonide, desoxymethasone, diflorasone diacetate, diflucortolone valerate, flumethasone pivalate, fluclorolone acetonide, fluocinolone acetonide, fluocionoide, fluocortin butyl, flucortolones, fluprednidene acetate, flurandrenolone, halcinonide, hydrocortisone, hydrocortisone acetate, hydrocortisone butyrate, methylprednisolone acetate, nometasone furoate, triamcinolone acetonide, and de-esterified base compounds, esters of base compounds, salts thereof and combinations thereof. A preferred corticosteroid is hydrocortisone or a pharmaceutically acceptable lower alkyl ester thereof Suitable NSAIDs include diclofenac, ibuprofen, acetylsalicylic acid, piroxicam, ketoprofen, felbinac, and benzylamine. Such NSAIDs may be present with or without a hydrocortisone-type anti-inflammatory.

Suitable anesthetics include the aminoacylanilide compounds such as lidocaine, prilocaine, bupivacaine, levo-bupivacaine, ropivacaine, mepivacaine and related local anesthetic compounds having various substituents on the ring system or amine nitrogen; the aminoalkyl benzoate compounds, such as procaine, chloroprocaine, propoxycaine, hexylcaine, tetracaine, cyclomethycaine, benoxinate, butacaine, proparacaine, butamben, and related local anesthetic compounds; cocaine and related local anesthetic compounds; amino carbonate compounds such as diperodon and related local anesthetic compounds; N-phenylamidine compounds such as phenacaine and related anesthetic compounds; N-aminoalkyl amide compounds such as dibucaine and related local anesthetic compounds; aminoketone compounds such as falicaine, dyclonine and related local anesthetic compounds; and amino ether compounds such as pramoxine, dimethisoquien, and related local anesthetic compounds; and para-amino benzoic acid esters such as benzocaine. Other suitable local anesthetics include ketocaine, dibucaine, amethocaine, propanacaine, and propipocaine. A preferred anesthetic is pramoxine.

In one embodiment, the active agent is an antiobiotic, particularly an antifungal agent. Suitable antifungal agents include clotrimazole, econazole, ketoconazole, itraconazole, miconazole, oxiconazole, sulconazole, butenafine, naftifine, terbinafine, undecylinic acid, tolnaftate, nystatin, and sertaconazole nitrate. Any conventional topical antibiotic can be used; for example, the antibacterial agent fusidic acid or the antiviral agent acyclovir.

C. Emulsion

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

d. Excipients

Buffers preferably buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, and thimerosal.

II. Method of Making the Formulation

a. Method of Preparing the Emulsion Concentrate

The oil phase is prepared by mixing together the surfactant(s) and emulsifier(s) and melting. The aqueous phase is prepared separately by dissolving the preservatives in water with heating. The aqueous phase is added to the oil phase with continuous high shear mixing to produce a milky emulsion. The emulsion is cooled and the pH is adjusted by the addition of a buffer.

The active agent can be either pre-dissolved in aqueous or organic phase or suspended/dispersed in the final emulsion.

The concentration of the surfactant(s) in the concentrate is from about 0.5 to about 5% by weight of the final composition. The concentration of the emulsifier(s) is from about 0.5% to about 5% by weight of the final composition. The concentration of the buffer(s) is from about 0.1% to about 5% by weight of the final composition and the concentration of the stabilizer(s) is from about 5% to about 15% by weight of the final composition.

Common formulation excipients and methods of making an aerosol foam can be found in Remington, The Science and Practice of Pharmacy (20^{th} Edition, Lippincott, Williams & Wilkins).

b. Method of Preparing the Formulation

The composition of the active agent is about 0.01% to about 30% by weight of the final composition. Specifically, the concentration of anti-inflammatories is from about 0.01% to about 10% by weight for corticosteroids and from about 0.1% to about 3% by weight for NSAIDs. The concentration of topical anesthetics is from about 1% to about 10% by weight and the concentration anti-fungals and other antibiotics is from about 0.3% to about 5% by weight. The topical anesthetic is preferably dissolved in the aqueous phase.

The emulsion concentrate is placed in pressure cans, preferably coated aluminum cans to prevent corrosion, such as epoxy-coated cans. The lid and dispensing apparatus are crimped in place. The can is charged with propellant to the stated level, for example, by adding 30 grams of propellant per 70 grams of emulsion. At the time of application, the mixture of the emulsion with the propellant may be insured by shaking, optionally with the aid of a mixing bead. The dispenser may be metered or unmetered (continuous). Metered dispensing is preferred for highly active materials such as hydrocortisone and other steroids. The can may be arranged for either “upside down” spraying with the valve at the bottom, or the can have a dip tube so that the foam can be sprayed while the can is upright with the valve at the top. The concentration of the HFA propellant(s) is from about 10% to about 60% by weight of the final composition, more preferably about 20% to about 50% by weight of the final composition. In a preferred embodiment, the emulsion concentrate is mixed with an HFA propellant so that the final formulation in an aerosol can comprises about 50% to about 80% of concentrate and about 20% to about 50% of propellant. In a more preferred embodiment, the final formulation in an aerosol can contain 70% concentrate and 30% propellant.

III. Mode of Administration

a. Method of Administration to a Patient

The formulation is administered to the skin or mucous membranes of a patient to treat a disease of the skin or mucous membranes. A selected amount of product is dispensed from the spray can, preferably onto the site to be treated. For non-critical active agents, the foam can be administered into the palm of the hand (the latter is also preferred when the application site in not visible). The amount to be delivered can be determined by the prescribing physician or as directed in the instructions for non-prescription products. Alternatively, a fixed dose using the metering dispenser can be administered. The foam is rubbed into the skin at the site to be treated. If contact with the hand is to be avoided, a glove may be worn; or, the foam may be left in place, wherein it will eventually collapse and deliver the active ingredient to the surface of the skin.

### EXAMPLES

### Example 1

### Topical Aerosol Foam for the Delivery of Hydrocortisone Acetate

A. Concentrate

| | | Preferred range, |
|---|---|---|
| INGREDIENT | Content, % (w/w) | % (w/w) |
| Hydrocortisone Acetate, USP | 1.0 | 0.5-5.0 |
| Propylene Glycol, USP | 10 | 5-15 |
| Cetyl Alcohol, NF | 0.70 | 0.5-1.5 |
| Triethanolamine NF | 0.10 | 0.01-1.0 |
| Steareth-10 | 0.50 | 0.25-1.5 |
| Emulsifying Wax, NF | 1.50 | 0.05-3.0 |
| Methylparaben | 0.11 | 0.05-0.15 |
| Proplyparaben | 0.03 | 0.02-0.05 |
| Water | 86.06 | 80-90 |
| TOTAL | 100 | |

1. The oil phase is prepared by mixing the cetyl alcohol, Steareth-10, and emulsifying wax and heating to 70-80° C. to melt.

2. The aqueous phase is prepared separately by dissolving the parabens in about 80% of the water listed above with heating to about 70 -80° C.

3. The aqueous phase is added to the oil phase with continuous high shear mixing to produce a milky emulsion.

4. The emulsion is cooled to about 30-40° C.; the emulsion thickens but remains a liquid.

5. The pH is adjusted if necessary by the addition of triethanolamine.

6. Separately, the hydrocortisone is suspended in propylene glycol and treated to eliminate any large aggregates. In a small scale operation, the mixture is milled. The final hydrocortisone particle size is small enough to allow aerosolization, for example, less than about 20 microns in diameter, preferably less than about 10 microns, more preferably, less than about 5 microns.

7. The hydrocortisone suspension is added to the emulsion with mixing.

8. The formulation is brought to the final weight with the addition of water.

The amount of triethanolamine is sensitive to the particular lots of ingredients, and the amount added determines the final pH of the product. The preferred pH in this formulation is about pH 4 to about 7.

B. Propellant

The concentrate is placed in an aerosol spray can, and the can is loaded with either isobutane-propane mixture or with HFA134a so that the composition is approximately 70% concentrate and 30% propellant (3 grams of propellant are added per 7 grams of concentrate).

### Example 2

### Topical Aerosol Foam for the Delivery of Lidocaine

A. Concentrate:

| | | Preferred range, |
|---|---|---|
| INGREDIENT | Content, % (w/w) | % (w/w) |
| Lidocaine, USP | 5.0 | 1.0-5.0 |
| Propylene Glycol, USP | 10.0 | 5-15 |
| Cetyl Alcohol, NF | 0.70 | 0.5-1.5 |
| Triethanolamine NF | 0.10 | 0.01-1.0 |
| Steareth-10 | 0.50 | 0.25-1.5 |
| Emulsifying Wax, NF | 1.50 | 0.05-3.0 |
| Methylparaben | 0.11 | 0.05-0.15 |
| Proplyparaben | 0.03 | 0.02-0.05 |
| Water | 82.06 | 80-90 |
| TOTAL | 100 | |

B. Propellant

The above formula is placed in an aerosol spray can, and the can is loaded with HFA134a so that the composition is approximately 70% concentrate and 30% HFA, i.e., 3 grams of propellant are added per 7 grams of concentrate.

The composition is mixed and dispensed essentially as described in Example 1. A formulation incorporating both an anti-inflammatory and an anesthetic would be useful in treating skin inflammatory conditions.

### Example 3

### A Topical Aerosol Foam for the Delivery of Itraconazole

A. Concentrate

| | | Preferred range, |
|---|---|---|
| INGREDIENT | Content, % (w/w) | % (w/w) |
| Itraconazole | 1.0 | 0.5-2.0 |
| Propylene Glycol, USP | 10 | 5-15 |
| Cetyl Alcohol, NF | 0.70 | 0.5-1.5 |
| Triethanolamine, NF | 0.10 | 0.01-1.0 |
| Steareth-10 | 0.50 | 0.25-1.5 |
| Emulsifying Wax, NF | 1.50 | 0.05-3.0 |
| Methylparaben | 0.11 | 0.05-0.15 |
| Proplyparaben | 0.03 | 0.02-0.05 |
| Water | 86.06 | 80-90 |
| TOTAL | 100 | |

B. Propellant

The above formula is placed in an aerosol spray can, and the can is loaded with HFA134a so that the composition is approximately 50% concentrate and 50% HFA, i.e., 5 grams of propellant are added per 5 grams of concentrate. The composition is mixed and dispensed essentially as described in Example 1.

Unless defined otherwise, all technical and scientific terms used herein have the same meanings as commonly understood by one of skill in the art to which the disclosed invention belongs. Publications cited herein and the material for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A topical foam aerosol formulation comprising
(a) an active agent or agents selected from the group consisting of anti-inflammatory agents, topical anesthetics, topical antibiotics, anti-fungal agents, and combinations thereof, solubilized or dispersed in an oil and water emulsion, wherein the emulsion does not contain volatile lower alcohols; and
(b) a propellant consisting essentially of a hydrofluoroalkane or a mixture of hydrofluoroalkanes, without additional co-solvents or co-propellants, contacting the emulsion to produce an immediate foaming action on expulsion from a pressurized container.

2. The formulation of claim 1 comprising a water-insoluble active agent in the oil phase and a water-soluble active agent in the aqueous phase.

3. The formulation of claim 2 wherein the active agent is an anti-inflammatory agent.

4. The formulation of claim 3 wherein the anti-inflammatory agent is selected from the group consisting of alclometasone dipropionate, amcinonide, beclamethasone dipropionate, betamethasone benzoate, betamethasone dipropionate, betamethasone valerate, budesonide, clobetasol propionate, clobetasone butyrate, desonide, desoxymethasone, diflorasone diacetate, diflucortolone valerate, flumethasone pivalate, fluclorolone acetonide, fluocinolone acetonide, fluocionoide, fluocortin butyl, flucortolones, fluprednidene acetate, flurandrenolone, halcinonide, hydrocortisone, hydrocortisone acetate, hydrocortisone butyrate, methylprednisolone acetate, nometasone furoate, triamcinolone acetonide, diclofenac, ibuprofen, acetylsalicylic acid, piroxicam, ketoprofen, felbinac, benzylamine, and combinations thereof.

5. The formulation of claim 3 wherein the concentration of the anti-inflammatory agent is from about 0.01% to 10%.

6. The formulation of claim 2 wherein the active agent is a topical anesthetic.

7. The formulation of claim 6 wherein the topical anesthetic is selected from the group consisting of lidocaine, prilocaine, bupivacaine, levo-bupivacaine, ropivacaine, mepivacaine, procaine, chloroprocaine, propoxycaine, hexylcaine, tetracaine, cyclomethycaine, benoxinate, butacaine, proparacaine, butamben, diperodon, phenacaine, falicaine, dyclonine, pramoxine, dimethisoquien, benzocaine, amethocaine, dibucaine, ketocaine, propanocaine, propipocaine, and combinations thereof.

8. The formulation of claim 6 wherein the concentration of the anesthetic is from about 1% to about 10%.

9. The formulation of claim 2 wherein the active agent is an antibiotic or antifungal agent.

10. The formulation of claim 9 wherein the active agent is an antifungal agent selected from the group consisting of clotrimazole, econazole, ketoconazole, itraconazole, miconazole, oxiconazole, sulconazole, butenafine, naftifine, terbinafine, undecylinic acid, tolnaftate, nystatin, and sertaconazole nitrate.

11. The formulation of claim 9 wherein the concentration of the antifungal or antibiotic agent is from about 0.3% to 5%.

12. A method of making a hydrofluoroalkane containing topical foam formulation free of volatile lower alcohols comprising
(a) making an oil in water emulsion with a predominantly, more than 50%, aqueous phase,
(b) either dissolving an active agent or agents selected from the group consisting of anti-inflammatory agents, topical anesthetics, topical antibiotics anti-fungal agents, and combinations thereof in the aqueous or oil phase prior to emulsification or adding non-water soluble, non-oil soluble drug to the emulsion to form a dispersion in the emulsion, and
(c) adding a propellant consisting essentially of a hydrofluoroalkane or a mixture of hydrofluoroalkanes, without additional co-solvents or co-propellants, to the emulsion to produce an immediate foaming action on expulsion from a pressurized container.

13. A hydrofluoroalkane containing topical foam formulation free of volatile alcohols produced by the method of claim 12.
